(19) United States
(12) Patent Application Publication
(10) Pub. No.: US 2024/0366062 A1
(43) Pub. Date: Nov. 7, 2024

(54) VISUAL ODOMETRY AND SEMANTIC SEGMENTATION IN HIGH-SPEED DETECTION OF ANOMALIES IN MEDICAL SCOPES

(71) Applicant: BH2 Innovations Inc., Newbury Park, CA (US)

(72) Inventors: Salmaan Hameed, San Jose, CA (US); Harut Margaryan, Yerevan (AM)

(21) Appl. No.: 18/646,460

(22) Filed: Apr. 25, 2024

Related U.S. Application Data
(60) Provisional application No. 63/464,482, filed on May 5, 2023.

(51) Int. Cl. A61B 1/00; G06T 7/00; G06T 7/10

(52) U.S. Cl. CPC A61B 1/000096; A61B 1/000094; G06T 7/0012; G06T 7/10; G06T 2207/10068; G06T 2207/20084

(57) ABSTRACT

AI-based methods detect and recognize surface anomalies along a channel of a medical scope from video taken by a camera pushed through the channel, and track the position of the camera in the channel. The image data output by the video camera is subjected to semantic segmentation to identify the surface anomalies, and is concurrently subjected to a feature detection algorithm to determine keypoints in frames of the image data. The keypoints can be used to update the camera position in the channel and can also be used to determine the distance of the surface anomalies from an end of the channel. Accordingly, the surface anomalies can be tagged so they are not counted as new anomalies when viewed again.

### CROSS REFERENCE TO RELATED APPLICATIONS

The present utility patent application is related to and claims the benefit of priority of U.S. Provisional Patent Application No. 63/464,482 filed on May 5, 2023 and titled “Advancements in the High Speed Detection and Characterization of Anomalies in Medical Scopes and the Predictability of Viability of Medical Scopes”, the entire contents of which are hereby incorporated by reference.

### BACKGROUND

### Field

The present technology relates to medical scopes such as endoscopes and the like. In particular, the present technology relates to the inspection of channels of medical scopes to detect and identify sources of contamination, structural defects, etc.

The present technology also relates to computer vision and, in particular, to image analysis and recognition using artificial intelligence, and to visual odometry.

### Description of Related Art

Rigid and flexible videoscopes are used in a wide array of industries to provide visual access to regions deep inside pieces of equipment. Chief among these industries is the field of internal medicine. Crucial to this field are medical scopes used to provide images of internal parts of the body for screening and making medical diagnoses, and which may additionally allow for therapeutic treatments to be carried out. With respect to the latter, medical scopes may have narrow working channels or “lumens” for containing accessories (medical instruments or manipulators) used to carry out procedures in minor surgeries. For example, endoscopes are a class of devices that allow parts of the body such as the colon (colonoscope), bladder (cystoscope), kidneys (nephroscope), bronchial tubes (bronchoscope), joints (arthroscope), thorax (thoracoscope), or abdomen (laparoscope) to be observed and treated, if necessary, in a minimally invasive way. Examples of the accessories that may be inserted into the body through working channels of endoscopes are forceps for conducting biopsies or removing polyps during a colonoscopy, electrodes for cauterizing tissue, cytology brushes to collect cells from the bronchi or gastrointestinal tract, aspiration needles for conducting biopsies, and instruments for placing elastic bands around bleeding veins (banding of esophageal varices).

Thus, although endoscopes vary widely in structure, they share some basic components. Specifically, and referring to FIGS. 1A and 1B, an endoscope 100 will typically include an insertion tube 110 (in this example, a flexible insertion tube) that is inserted through an opening in the body, a control section 120 to which the insertion tube 110 is connected and by which various parts of the endoscope 100 are controlled by the operator, a connector section 130 connected to the control section 120 for connecting various external devices to the endoscope 100 and an optical system generally designated by reference numeral 140.

The optical system 140 is composed of a light guide extending through the insertion tube 110 to a distal end 110a of the insertion tube 110 and connected to a light source for illuminating the region of interest inside the body, and an image guide (not shown) extending through the insertion tube 110 to an objective lens 112 at the distal end 110a. The objective lens 112 captures light from the illuminated region of interest and focuses the light back onto the image guide for producing an image of the region of interest. The light and image guides may be made up of fiberoptics only a few millimeters in diameter to transfer illumination from the light source in one direction and images in real time in the other direction. The light source is not shown as it is typically not part of the scope itself. Likewise, although some endoscopes include an eyepiece optically connected to the image guide and through which the images of the region of interest can be viewed, in this example the connector section 130 is connected to a display upon which the images of the region of interest can be viewed.

Still referring to FIGS. 1A and 1B, the insertion tube 110 further includes an instrument channel 114 into which one of the accessories may be inserted and withdrawn by way of a channel opening 116 in the insertion tube 110. The instrument channel 114 is a narrow duct surrounded by opaque material of the insertion tube 110. Other working channels in the insertion tube may include an air/water pipe(s) 116 for use in providing air, suction and/or irrigation to the region of interest. A working channel, e.g., the instrument channel 114, may have a distal end open at the distal end 110a of the insertion tube 110, as shown in FIG. 1B, and a proximal end open at the “channel opening” shown in FIG. 1A.

Since the components and features of medical scopes such as endoscopes are generally well-known in the art, the example shown in FIGS. 1A and 1B will not be described in further detail for the sake of brevity.

The insertion tubes of medical scopes may contact a source of contamination such as a mucous membrane during a clinical procedure. Accordingly, medical scopes are potential sources of infection during subsequent uses. Therefore, at the conclusion of each use, medical scopes are “reprocessed” in an attempt to eliminate all microorganisms from the scopes. The microorganisms may reside in stains and droplets of moisture, blood, lubricant, etc. along a channel of the scope. The reprocessing of endoscopes basically includes a manual washing process, a high-level disinfecting (HLD) process carried out with a liquid chemical germicide, and a drying process during which the scope is hung in storage for some period of time. The manual washing process is typically an enzymatic cleaning process.

Medical scopes may also suffer occult damage during use and/or over time. For example, the surface defining the instrument channel of the scope may be scratched as an instrument is inserted or withdrawn along the channel and especially along a curved portion of the channel. The layer of material that defines the channel within the insertion tube may exhibit peeling. Both of these types of defects provide locations at which debris may reside and harbor microbes and where the efforts of reprocessing to remove the debris is resisted. And in flexible scopes in particular, the insertion tube may be bent or even bitten by a patient to such an extent as to crimp or buckle the instrument channel. This not only increases the likelihood of scratching or peeling (at the location of the crimp) but may give rise to mechanical breakdown of the scope. In some cases, the crimping or buckling causes operational failure during a clinical procedure with resulting serious harm to the patient undergoing the procedure.

Because mechanical breakdown and infections have been linked to occult damage, droplets and other residue remaining in the channel, it is standardized practice to inspect endoscopes frequently for these and other types of “surface anomalies” in the channels. Results of the inspection process are used for quality assurance and risk management and infection control. Specifically, the inspection process is used to determine whether the medical scope must be reprocessed again, sent out for repairs or even discarded as being at the end of its useful life. Industry standards and guidelines from manufacturers of endoscopes recommend the use of borescopes for this purpose. A borescope is an optical tool at the end of either a rigid or flexible tube and similarly to an endoscope, constitutes a remote visual inspection device.

Referring to FIG. 2, a typical digital borescope 200 for use in inspecting the narrow channels of medical scopes, such as instrument channel 114 of the endoscope 100 shown in FIGS. 1A and 1B, has a light guide 212 extending through the tube to a light source for illuminating the channel when in use, an image sensor 214, an objective 216 for focusing light onto the image sensor 214, and a signal line (bus) 218 for transmitting video signals (digital image data) output by the image sensor 214. The video signals are processed into images of the channel and displayed on the display of a custom or computer monitor, smartphone, etc. connected to signal line 218. The image sensor 214 of the borescope 200 may thus be considered as a “video camera”. The images sensor 214 of this example comprises a CMOS device provided on a chip. In some examples, the video camera can be mounted so as to rotate as it is translated longitudinally. Due to size constraints dictated by the narrowness of the channel of the medical scope being inspected, the pixel array of the CMOS device may have no more than 1600 pixels, e.g., may be a 200×200 or 400×400 array. Thus, borescope 200 may produce only standard definition as opposed to high definition (HD) images.

Therefore, the personnel (e.g., endoscopists) who are charged with visually inspecting and real time evaluation of medical scopes using a borescope require highly specialized training individualized throughout even similar departments or facilities. Nonetheless, the inspection process has a degree of uncertainty based on human error inherent in the inspection process. To minimize human error, such personnel must push a borescope slowly through the channel of a medical scope, i.e., to ensure that they do not overlook and are capable of identifying surface anomalies that require further attention. Note here that (the video camera of) the borescope may be inserted into the channel from either the distal or proximal end to carry out the inspection process.

Furthermore, facilities such as large hospitals may carry out scores of procedures using medical scopes on daily basis. The time and hence, labor costs, required to inspect these scopes periodically and especially after each use as recommended is thus enormous.

Artificial intelligence (AI) is a developing technology in the field of image recognition and has been used to detect boundaries of features or objects in digital images and recognize the features or objects from the boundaries. A goal of applying AI-based technology to the inspection of channels of medical scopes would be to minimize human error with the ultimate goal being unsupervised medical scope evaluations.

One “proof of concept” study by Barakat et al. at Stanford Medical Hospital showed promise for the ability of AI technology to recognize the presence of surface anomalies like scratches, peeling, and droplets, in images of channels of endoscopes. This study was performed using an object detection model of localizing the surface anomalies in bounding boxes, respectively, and identifying each anomaly in its bounding box. However, the object detection model used in this study has various shortcomings: the model is rather incapable of detecting very fine surface anomalies (high sensitivity), characterizing surface anomalies with a high degree of specificity, and ensuring reliable predictive power (accuracy) for different scopes. Predictions would also have to provide for an assessment of the severity of the anomalies, both individually and collectively. Furthermore, handling the speed at which a borescope could be pushed through a channel of a medical scope when attempting to carry out inspections rapidly would also require the images of the borescope to be processed at a high speed to ensure that surface anomalies were detected with the necessary degree of quality assurance.

However, providing many or all of these requirements is either unsurmountable through the use of conventional AI image-recognition techniques such as those based on object detection, or requires high computational power resulting in an expectedly low corresponding processing speed.

Therefore, there remains a need for improved methods and systems that can detect anomalies along a channel of a medical scope at a relatively high speed and, in particular, without sacrificing other advantages such as sensitivity and reliability. Similarly, there remains a need for AI image recognition methods and systems capable of rapidly detecting anomalies along a channel of a medical scope in real time, and even as quickly as a digital borescope can be pushed by a technician or device along the channel.

Moreover, the current results of visual inspection of the channels of medical scope which can be achieved are rather rudimentary and cannot be readily parlayed into high level information. Basically, the results do not readily allow for the anomalies to be viewed again or re-inspected. There also remains a need for methods by which the anomalies along a channel of a medical scope can be tagged in such a way that their relative position is known.

### SUMMARY

According to one aspect of the disclosed technology, there is provided a method of detecting anomalies along a channel of a medical scope. The method includes accessing, at an artificial intelligence (AI) image recognition system including a deep learning tool, image data produced by a digital borescope as the borescope is pushed along a length of a channel of a medical scope, predicting in real time as the digital borescope is pushed through the channel of the medical scope, the presence of one or more types of anomalies at locations along the length channel, and providing an output of indicia of surface anomalies along the length of the channel. The image data produced from pixels of an image sensor of the borescope is using the deep learning tool including by a convolutional neural network trained on a data set of images of surface anomalies along channels of medical scopes of a kind like that through which the borescope is pushed. The convolutional neural network is configured for semantic segmentation. The semantic segmentation occurs along only a context path in the AI image recognition system once the image data is accessed by the system.

According to a similar aspect, there is provided a method for use in evaluating medical scopes, including pushing a digital borescope along a length of a channel of a medical scope to capture images of the channel and output image data representative of the images, transmitting image data from each of pixels of an image sensor of the digital borescope to an artificial intelligence (AI) image recognition system including a deep learning tool to predict the presence of at least one type of anomaly at locations along the length channel of the medical scope, and analyzing indicia of anomalies along the channel from output of the image recognition system. The deep learning tool has a convolutional neural network trained on a data set of images of surface anomalies along channels of medical scopes of a kind like that through which the borescope is being pushed. The deep learning tool is configured for semantic segmentation and to process in real time image data produced from pixels of an image sensor of the borescope, and the AI image recognition system is configured to output indicia of surface anomalies along the length of the channel based on results of the processing of the image data by its deep learning tool. The semantic segmentation is carried out along only a context path in the AI image recognition system.

According to yet another similar aspect, there is provided an artificial intelligence (AI) image recognition system for use in detection of surface anomalies along a channel of a medical scope. The system includes a video input output (I/O) operative to receive data of video images, image processing deep learning modules operatively connected to the video input output (I/O) to receive data of video images therefrom, and a graphics output module including a video input output (I/O) operatively connected to the image processing modules to receive results of the processing of data of video images by the image processing modules and output the results. The deep learning modules are configured to process image data produced from an image sensor of a digital borescope, and include a convolutional neural network trained on a data set of images of surface anomalies present along a surface of channels of medical scopes and configured for semantic segmentation. The convolutional neural network is configured for semantic segmentation along only a context path once data of video images is output from the video I/O and received by the deep learning modules.

The use of a context path only for the semantic segmentation allows for especially high-speed processing of the video image data produced by a borescope without loss in detection accuracy, when applied to recognizing surface anomalies along the channels of medical scopes. Exemplary of this speed is a rate of about 0.03 seconds per frame.

According to still another aspect of the present technology, there is provided a method for use in evaluating a state of a medical scope having a working channel. The method includes accessing, at an artificial intelligence (AI) image recognition system including a deep learning tool, image data of video of the working channel of a medical scope produced by a video camera as the video camera is pushed along a length of the working channel beginning at an end of the working channel. The deep learning tool has a convolutional neural network configured for semantic segmentation along a context path, the convolutional neural network being trained on a data set of images of at least one type of surface anomaly along channels of medical scopes similar to that through which the borescope is pushed. Furthermore, the image data using the AI image recognition system is processed to both detect surface anomalies of said at least one type at locations along the length of the working channel and calculate for each of the surface anomalies detected an estimation of a distance between the detected surface anomaly and said end of the working channel. The processing includes semantic segmentation of the image data by the convolutional neural network along only the context path in the AI image recognition system once the data of video images is accessed by the AI image recognition system to detect the presence of surface anomalies along the working channel, extracting keypoints of images taken by the video camera within the working channel, and using the keypoints to determine amounts of translation of the video camera within the working channel. Still further, data of the types of surface anomalies detected and their relative locations within the working channel are produced based on results of the processing.

According to still another aspect of the present technology, the method for use in evaluating a state of a medical scope having a working channel includes pushing a video camera, including an image sensor having an array of pixels, through an end of the working channel and along a length of the working channel to capture images of a length of the working channel and output image data representative of the images, and transmitting image data from each of the pixels of the image sensor of the video camera to an artificial intelligence (AI) image recognition system including a deep learning tool having a convolutional neural network. The convolutional neural network is trained on a data set of images of surface anomalies along channels of medical scopes similar to that through which the video camera is being pushed, and the deep learning tool is configured for semantic segmentation along a context path. Furthermore, the image data produced from the pixels of the image sensor of the video camera is processed using the AI image recognition system to both detect surface anomalies along the length of the working channel and calculate for each of the surface anomalies detected an estimation of a distance between the detected surface anomaly and said end of the working channel. The processing includes semantic segmentation of the image data by the convolutional neural network along only a context path in the deep learning tool of the AI image recognition system once the image data is received by the AI image recognition system to detect the presence of surface anomalies along the working channel, extracting keypoints of images taken by the video camera within the working channel, and using the keypoints to determine amounts of translation of the video camera within the working channel. And still further, data of the types of surface anomalies detected and their relative locations within the working channel are produced based on results of the processing.

### BRIEF DESCRIPTION OF THE DRAWINGS

These and other objects, features and advantages of the present technology will be better understood from the detailed description of preferred embodiments and examples thereof that follows with reference to the accompanying drawings, in which:

FIG. 1A is a perspective view of an example of a medical scope to which the present technology can be applied;

FIG. 1B is an enlarged view of a distal end of an insertion tube of the medical scope;

FIG. 2 is a cross-sectional view of an example of a borescope to which the present technology can be applied;

FIG. 3 is a schematic diagram of an example of a network including an artificial intelligence (AI) image recognition system according to the present technology;

FIG. 4 is a block diagram of a portion the deep learning tool of the (AI) image recognition system of FIG. 3, with associated representations of image frames produced as a result of processing by modules and algorithms of the deep learning tool;

FIG. 5 is a schematic diagram of an Image Enhancement module of the deep learning tool;

FIG. 6A is a schematic diagram of real-time semantic segmentation network architecture of the AI image recognition system of FIG. 3;

FIG. 6B is a schematic diagram of the attention refinement module of the of the semantic segmentation network architecture;

FIG. 7 is a schematic diagram of an example of the backbone of the semantic segmentation network architecture shown in FIGS. 6A and 6B;

FIG. 8 is a process flow diagram of an example of a method of detecting anomalies along a channel of a medical scope according to the present technology;

FIGS. 9A, 9B, 9C, 9D, 9E and 9F are each a pair of images, one being an actual original image of the instrument channel of an endoscope captured by a digital borescope and the other being an image containing indicia of surface anomalies derived from the original image using an embodiment of the present technology as shown and described with reference to FIGS. 1-8;

FIG. 10A-10C show an example of a visual odometry process (or tool) by which the relative position of a video camera within the working channel of a medical scope can be determined based on image data being captured by the camera according to the present technology, wherein FIG. 10A is a flow diagram of the visual odometry process, FIG. 10B is an example of an actual image of a working channel of a medical scope showing keypoints detected by the ORB algorithm in the process, and FIG. 10C are examples of current and previous frames showing the distance calculated of the amount the video camera was translated between positions of the frames; and

FIG. 11 is a schematic diagram of a method by which the visual odometry process of FIG. 10A can be adapted to identify/record the locations of surface anomalies along a surface of a working channel of a medical scope, according to the present technology.

### DETAILED DESCRIPTION

Embodiments of the present technology and examples thereof will now be described more fully in detail hereinafter with reference to the accompanying drawings. In the drawings, elements may be shown schematically for ease of understanding. Also, like numerals and reference characters are used to designate like elements throughout the drawings.

Certain examples may be described and illustrated in terms of blocks which carry out a described function or functions. These blocks, which may be referred to herein as modules or the like, are physically implemented by analog and/or digital circuits such as logic gates, integrated circuits, microprocessors, microcontrollers, memory circuits, passive electronic components, active electronic components, optical components, hardwired circuits and the like, and may be driven by firmware and/or software of non-transitory computer readable media (CRM). In the present disclosure, the term non-transitory computer readable medium (CRM) refers to any medium that stores data in a machine-readable format for short periods or in the presence of power such as a memory device or Random Access Memory (RAM). The circuits may, for example, be embodied in one or more semiconductor chips, or on substrate supports such as printed circuit boards and the like. The circuits constituting a block may be implemented by dedicated hardware, or by a processor (e.g., one or more programmed microprocessors and associated circuitry), or by a combination of dedicated hardware to perform some functions of the block and a processor to perform other functions of the block. Each block of the examples may be physically separated into two or more interacting and discrete blocks. Conversely, the blocks of the examples may be physically combined into more complex blocks while still providing the essential functions of the present technology.

The terminology used herein for the purpose of describing particular embodiments of the present technology is to be taken in context. For example, the term “comprises” or “comprising” when used in this disclosure indicates the presence of stated features or steps in a process but does not preclude the presence of additional features or steps. The term “about 1600 pixels” is to be understood as referring to pixel numbers of an array that can output standard definition (SD) as opposed to high-definition (HD) images. Standard definition images are generally understood in the art as no more than (480p) and thus, the term no more than 1600 pixels may refer to 400×400 or 200×200 pixel arrays. The term “about 0.03 seconds per frame” is used to encompass the practical working range of high speeds at which the disclosed examples of a deep learning tool according to the present technology can process the described video input data and output image recognitions results. Therefore, the term “at most about 0.03 seconds per frame” encompasses all practical higher speeds. The term “context path” is a term of art understood as a path along which global context information as distinguished from spatial information (information about spatially proximate pixels) is transcribed onto feature masks. The term “working channel” of a medical scope refers to any channel in a medical scope open at a distal end (at least) of the scope and which provides some key function during use, such as for use in guiding an instrument through the scope, in allowing a target area to be irrigated, etc.

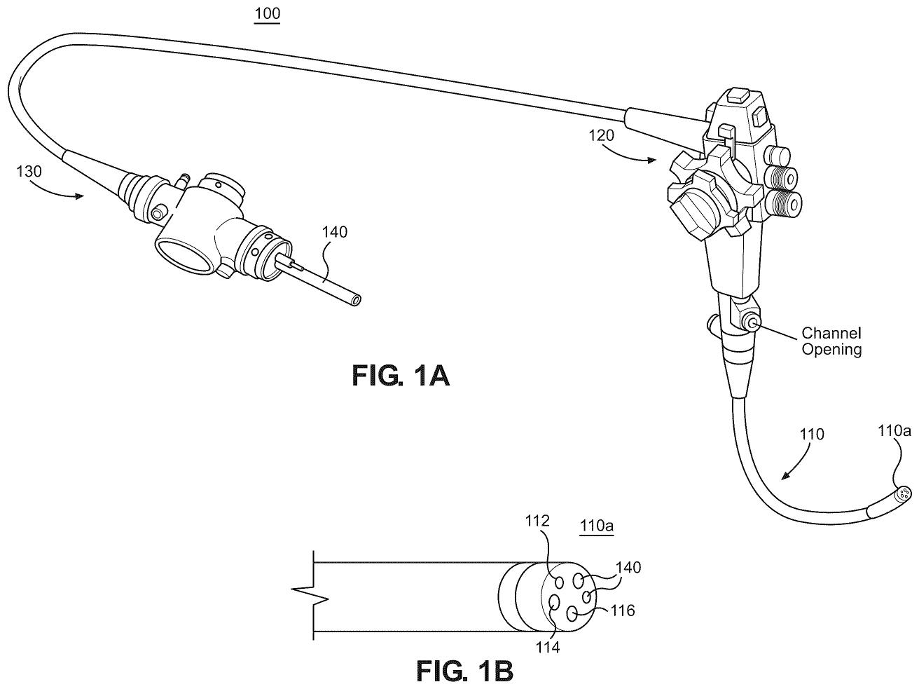

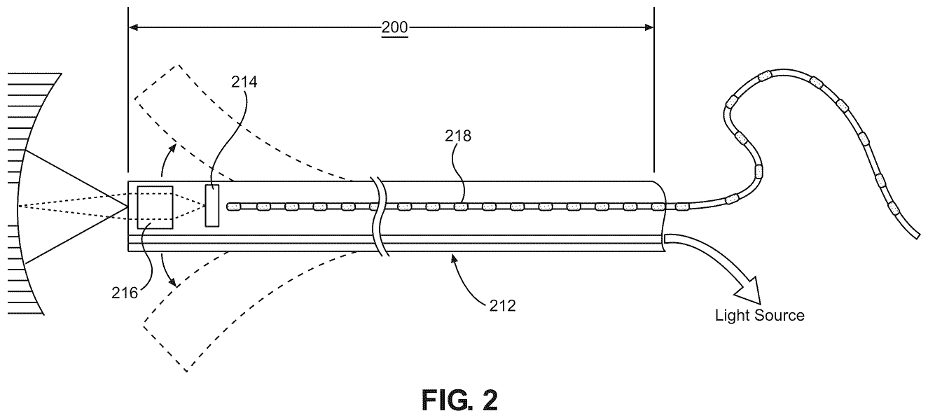

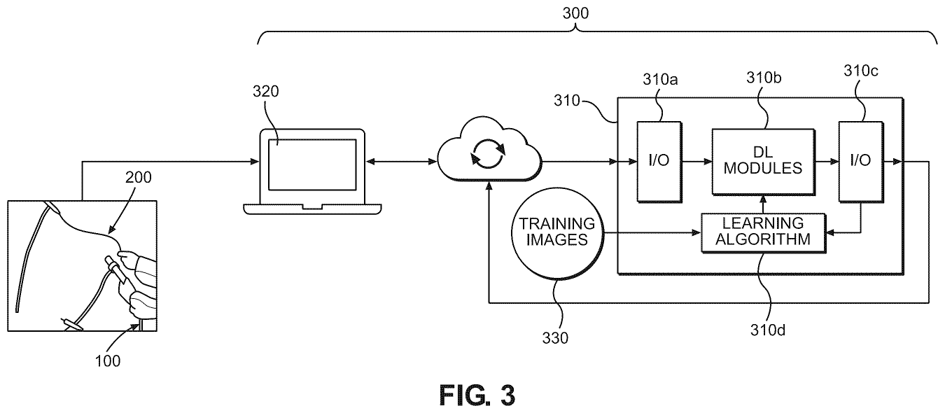

FIG. 3 illustrates a system or network in which surface anomalies along a channel of a medical scope are detected. The block at the left-hand side of FIG. 3 represents an environment, such as an SPD of a hospital, in which a medical scope 100 is inspected by a technician, e.g., a trained endoscopist, using a digital borescope 200 (FIGS. 1A, 1B and 2). An artificial intelligence (AI) image recognition system 300 may be operatively connected to the digital borescope 200 for real time detection of anomalies along a channel, e.g., an instrument channel, of the medical scope 100.

The AI image recognition system 300 includes a deep learning tool 310 and may also include a visual display unit 320 (typically, an LCD as a standalone or part of a computer such as a desktop or laptop). The visual display unit 320 may have a graphical user interface (GUI) by which a user of the present technology may call up images and indicia of surface anomalies output by the deep learning tool 310. The AI image recognition system 300 may be part of an enterprise system in which the components are connected to form a local area network (LAN) or carried out over the internet to provide a Service as a Service (SaaS). Therefore, the deep learning tool 310 may be provided on a server or the like, accessed by means of the web (as shown) or by an ethernet cable, router or hub, etc. Instructions for operating system AI image recognition system 300 including executing processes of the deep learning tool 310 are provided on non-transitory computer readable media (CRM).

The deep learning tool 310 comprises a graphics processor unit (GPU), i.e., a specialized processor. The deep learning tool 310 may also include associated memory and a CPU for executing algorithms in the AI image recognition system 300. The GPU of deep learning tool 310 is exemplified as including a digital video input output (I/O) 310a, deep learning modules 310b operatively connected to the digital video I/O 310a, and a graphics module (I/O) 310c operatively connected to the deep learning modules 310b. The deep learning tool 310 also has a training module 310d having an associated memory device(s) and is configured with a deep learning algorithm for supervised or semi-supervised training of the network implementing the deep learning modules 310b. In this example, deep learning module 310d is operatively connected to a memory 330 for storing data sets of training images, namely, images of channels of medical scopes including surface anomalies. The memory 330 may be separate from or an integral part of the deep learning tool 310. In an example of the present technology, the deep learning modules 310b comprise a residual learning framework and thus, are configured for residual learning. The digital video I/O 310a may comprise a digital video interface (DVI) or any type of video interface that can receive RGB video signals of image frames captured by the image sensor 214 of the digital borescope 200 and output the signals to the specialized GPU 310b. The graphics output module 310c may comprise a digital video I/O such as a digital video interface (DVI) or any known type of video interface that can receive video image signals from the specialized GPU 310b and a graphic generator that can generate graphics data from the image signals. The graphics output module 310c outputs the signals of the images produced by the GPU constituting the deep learning tool 310, along with indicia of the surface anomalies to the visual display unit 320.

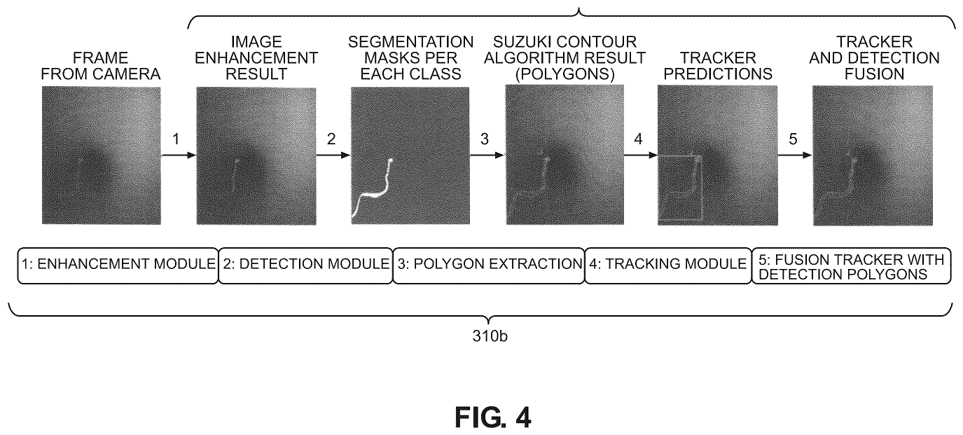

The deep learning modules 310b, and the algorithms executed on outputs produced therefrom, will be described in more detail with reference to FIG. 4. In the description that follows, for simplicity the term “image” or “video” may at times be used to refer to digital data representative of the image or video. Also, in FIG. 4, a representative image frame from image sensor 214 of the digital borescope 200 (“Frame from camera”), showing a surface anomaly (scratch) along the channel of the medical scope 100, is used for purposes of description but the same description applies to detecting and recognizing other types of surface anomalies according to the present technology.

As mentioned above, the image data from the pixels of the image sensor 214 (RGB color images) may be in standard definition format. The image data is input to the deep learning modules 310b by way of video I/O 310a. The image can be processed by the deep learning modules 310b at a rate as high as 0.03 frames per second, whereas humans require ¼ of a second to process one such image frame by eye. Or, put another way, the deep learning modules 310 configured according to the present technology can process an image captured by a digital borescope in no more than 30 milliseconds.

To this end, according to one embodiment of the present technology, the deep learning modules 310b comprise an image Enhancement module, a Detection module, and a Tracking Module and a Polygon extraction module, and a processor constituting modules for executing a Polygon extraction algorithm and a data Fusing algorithm. These modules/algorithms are described in detail below.

### 1: Enhancement Module

The Image enhancement module is configured to enhance the RGB color images produced by the digital borescope 200 to emphasize any surface anomaly in the images. In an embodiment of the present technology, the Image enhancement module comprises a digital filter selected to transform the images captured by the borescope 200, and accessed by the deep learning tool 310 through its digital video I/O 310a, into enhanced images. In one example, the Image enhancement module consists of a Bilateral filter for image smoothing, followed by a 2Dfilter for image sharpening, and finally a second Bilateral filter for image smoothing. Suitable examples of the 2D and Bilateral filters may be selected from those available in the open source computer vision library (OpenCV library), essential features of which are provided below.

### 2DFilter

A suitable example of the digital filter is a 2Dfilter is characterized by the following parameters, filter coefficients and correlation function:

Parameters

| | |
|---|---|
| src | input image. |
| dst | output image of the same size and the same number of channels |
| | as src. |
| ddepth | desired depth of the destination image. |
| kernel | correlation kernel, a single-channel floating point matrix for |
| | the RGB channel |
| anchor | anchor of the kernel that indicates the relative position of a |
| | filtered point within the kernel; the anchor should lie within |
| | the kernel; default value (−1,−1) means that the anchor |
| | is at the kernel center. |
| delta | optional value added to the filtered pixels before storing them |
| | in dst. |

Programming in Python, Input array (src), Output Array (dst), Input Array (kernel), and Point (anchor)
cv.filter2D (src, ddepth, kernel[, dst[, anchor[, delta [,borderType]]]]->dst, and
Correlation function dst(x,y)=Σ0≤x′<kernel.cols0≤y′<kernel.rowskernel(x′,y′)*src(x+x′−anchor.x,y+y′−ancho r.y)

The function may use the direct algorithm for small kernels.

### Bilateral Filter

Bilateral filtering is applied to the original image and then again to the image sharpened by the 2DFilter to suppress noise in the images. The concept behind bilateral filtering is described in detail on the homepage for the university of Edinburgh, School of Informatics, titled, “Bilateral Filtering For Gray and Color Images.”

A suitable example for each of the Bilateral filters is characterized by the following parameters and filter coefficients:

| | |
|---|---|
| src | floating-point, 3-channel image. |
| dst | Destination image of the same size and type as src. |
| d | Diameter of each pixel neighborhood that is used during |
| | filtering, computed from sigmaSpace. |
| sigmaColor | Filter sigma in the color space. |
| sigmaSpace | Filter sigma in the coordinate space. |
| borderType | border mode used to extrapolate pixels outside of the |
| | image. |

### Programming in Python

cv.bilateralFilter(src, d, sigmaColor, sigmaSpace[, dst[, borderType]])->dst

### 2: Detection Module

The Detection module has a convolutional neural network configured to apply a semantic segmentation process to the enhanced image output from the Enhancement module. In general, the input channel is the enhanced RGB image and has pixels correlating to those of the image output by the image sensor 214 of borescope 200. The Detection module assigns semantic labels (sematic context information) to pixels of the enhanced image. More specifically, the Detection module produces a (binary) semantic segmentation mask for each of several classes of surface anomalies, so that the pixels in each of the segmentation masks are encoded with binary data correlated with each class of surface anomaly. In the present embodiment, there are five (5) distinct classes of surface anomalies: droplet, peeling, crimp, scratch and stain. Therefore, in this example, the output of the Detection module is a binary mask with five (5) channels. Surface anomalies and parts thereof may be referred to hereinafter as “objects”.

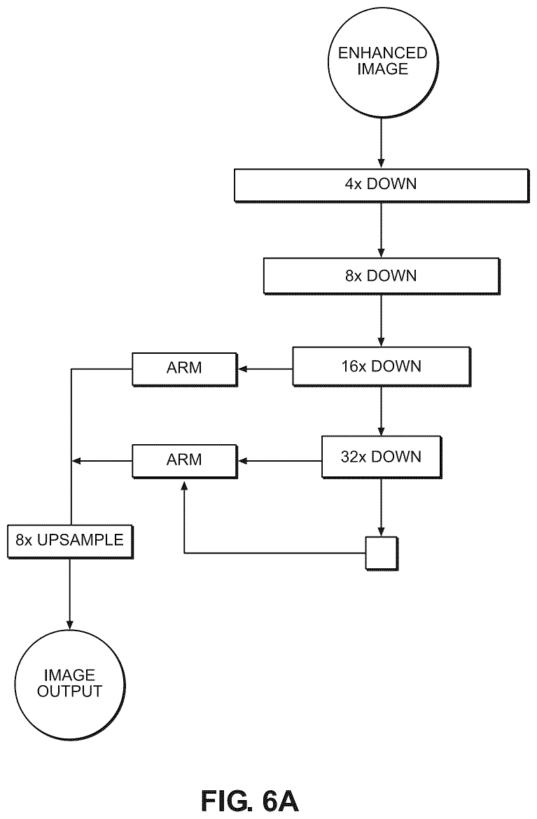

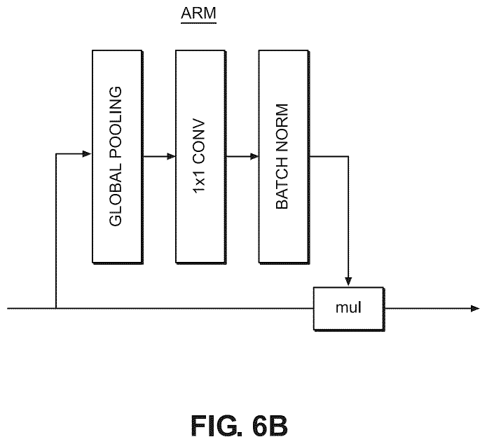

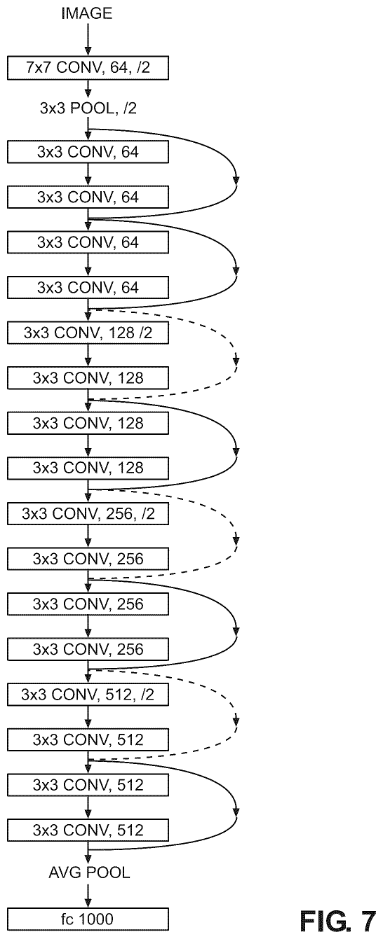

FIGS. 6A, 6B and 7 constitute a workflow diagram of the Detection module, with FIGS. 6A and 6B showing the workflow and FIG. 7 showing an example of the backbone of the network architecture by which the Detection module is implemented. The network architecture of the Detection module requires the highest computational power in the deep learning tool 310. The architecture of this example is designed to minimize the computational power and facilitate the high-speed processing by the deep learning tool 310. According to the present technology, the standard definition images output by a borescope as it is pushed rapidly through a channel of a conventional medical scope can be processed at a rate of about 0.03 seconds per image frame or less, without loss in ability to accurately predict and identify surface anomalies along the channel.

As can be seen from FIGS. 6A and 6B, the process is an end-to-end process having only relatively narrow channels with deep layers to encode semantic labels to a receptive field, namely, the region in the enhanced image that has an object. There is no encoding of spatial information, i.e., information of the relative position (distance and orientation) of pairs of pixels in the output of the Enhancement module. The semantic segmentation takes place only along a context path in the AI image recognition system 300.

Initially, the output of the Enhancement module (Module 1) is downsampled quickly to obtain a sufficient receptive field. Then, upsampling occurs directly on the same context path after using an attention refinement module (ARM), shown as two blocks in this figure. The attention refinement module (ARM) encodes output features into vectors by global average pooling to capture global context, thus refining the output at each of various stages of downsampling along the Path. To this end, the attention refinement module (ARM) is configured to execute in the following sequence a global average pooling of downsampled image data, a 1×1 convolution operation, batch normalization, a sigmoid function, and a matrix multiplication function.

The convolutional neural network is thus configured to downsample image data in a series of downsampling processes, apply global average pooling at a tail end of the downsampling processes, and subsequently upsample only an output derived from the global pooling.

The size of the enhanced image input to the Detection module by the Enhancement module (Module 1) is relatively small. Thus, the present inventors have found that it becomes unnecessary to encode spatial information (information about the relative location and orientations of pairs of pixels) into the output of the Detection module to preserve accuracy in the results of the detection of objects by the module.

This embodiment of the present technology can be characterized as essentially consisting of local operations, namely convolutions, pooling, sampling. Thus, computational power is kept to a minimum.

ResNet architecture is used as the backbone to produce semantic edge maps. Here, the training provided by the ResNet architecture increases the effective receptive field.

There are many variants of ResNet architecture, but according to an embodiment of the present technology the Detection module is implemented using ResNet-18 (an 18-layer ResNet). The essential features of ResNet-18 architecture, for use in implementing the present embodiment, are shown in FIG. 7. This baseline architecture is known, per se, and the concepts behind it, such as the use of skip connections shown in the figure, are described in Deep Residual Learning for Image Recognition, He et al., arxiv.org/abs/1512.03385 (December 2015), the entire contents of which are hereby incorporated by reference. Therefore, such architecture will not be described herein in more detail.

### 3: Polygon Extraction

Objects of the same class in an image cannot be distinguished from each other using the semantic segmentation model/step provided in the Detection module. That is, the semantic segmentation model executed by the Detection module produces masks in which objects in the same class are not distinguished from each other.

The Polygon extraction module uses the segmentation masks produced in the semantic segmentation step to extract polygons which the capture the geometry of each discrete object in the image output by the Detection module. Therefore, in the algorithm executed by the Polygon extraction module, the edges of the objects are integrated with polygons to determine the type of surface anomaly.

To this end, the Polygon extraction module executes a border-following algorithm. In this process, polygons which bound the objects (surface anomalies) are extracted from the images, in contrast to bounding boxes used in more conventional object detection AI techniques. In an embodiment of the present technology, the GPU of the deep learning tool 310 is configured with a Suzuki contour algorithm to implement the Polygon extraction module. The algorithm can be programmed using Python provided in the popular OpenCV library under the topic heading, “Finding contours in your image”

### Programming in Python

(#Find contours, hierarchy=cv2.findCountours(binary mask, cv2.RETR_TREE, cv2.CHAIN_APPROX_SIMPLE)).

In the illustrated example, the polygon extraction process identifies the anomaly as a scratch along the surface of channel 114.

### 4: Tracking Module

Module 4 is a Tracking module (which may be referred to as a “tracker” hereinafter) that tracks results of the semantic segmentation and polygon extraction processes to identify unique objects in the video image data and maintain their trajectory in the stream of images constituting the video image data. In this way, the existence of discrete occurrences of a particular type of surface anomaly can be determined. In the illustrated example, whether the edges form one continuous scratch or more than one discrete scratch can be determined.

The Tracking module of the present technology is configured to execute a ByteTrack data association method for the pipeline of images processed by the modules 310b of the deep learning tool 310. Modern tracking models use a “tracking-by-detection” framework, which first uses a detection model to obtain the bounding boxes (bboxes) of objects, and then updates the tracker state based on the bboxes. In the Tracking module of an embodiment of the deep learning tool 310 according to the present technology, bboxes of polygons obtained from the Polygon extraction module are used instead. The ByteTrack model accesses the bboxes as input, as well as confidence scores for each bbox that the Detection module produces. The semantic segmentation process performed by the Detection module predicts the probability (confidence score) for each pixel separately, so by default confidence scores are not derived for each object. Rather, the present technology takes the probability matrix of each class and crops inside each polygon of the object. Then the average value of the probabilities inside the polygon are calculated, thus obtaining the confidence score for that particular object.

To this end, the ByteTrack-based tracker of the Tracking module combines a notion model with a Kalman Filter that manages a queue (tracklets) to store objects being tracked, and performs tracking and matching between bounding boxes with low confidence scores.

The Tracking Module of this example of the present technology also executes the following processes or subroutines:
1. Polygon adjustments.
2. Restoring polygons if for some reason the execution of the semantic segmentation process by the Detection module did not detect objects, i.e., surface anomalies present along the channel.
3. Extracting frames from the video image data in which different objects are present and generate reports.

### 5: Fusing

A fusion algorithm is executed on the output of the Tracking module to blend the output of the detection with the results of the Tracking module for the purpose of counting and keeping track of the type of individual objects (surface anomalies). In essence, Module 5 is designed to track objects over time in a video sequence. The algorithm uses the output bounding box from the object detector as an initialization and then refines it over subsequent frames based on the object's motion and appearance. This means that the output bounding box from the tracker may differ from the one that came from the object detector due to changes in the object's location, scale, or orientation over time. Therefore, it may be necessary to adjust the polygon of the object to match the bounding box from the tracker.

More specifically, the vertices of the polygon are adjusted to refine the polygon so that it fits within the bounding box. The polygon is scaled and translated to match the dimensions and position of the bounding box. A step-by-step approach follows:
1. Compute the minimum bounding box that encloses the polygon.
2. Compute the scaling and translation factors needed to map the minimum bounding box to the output bounding box from the tracker.
3. Apply the scaling and translation factors to each vertex of the polygon to obtain the refined polygon.

This increases the success rate of the deep learning tool 310 in recognizing and discriminating surface anomalies from one another.

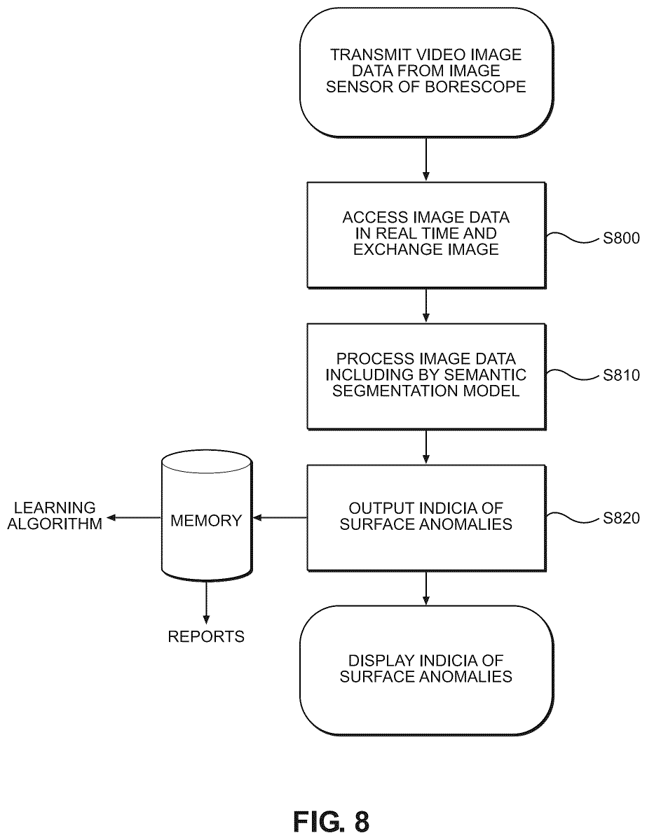

FIG. 8 shows an example of a method of detecting anomalies along a channel of a medical scope using the deep learning tool 310 shown in and described with reference to FIGS. 3-6.

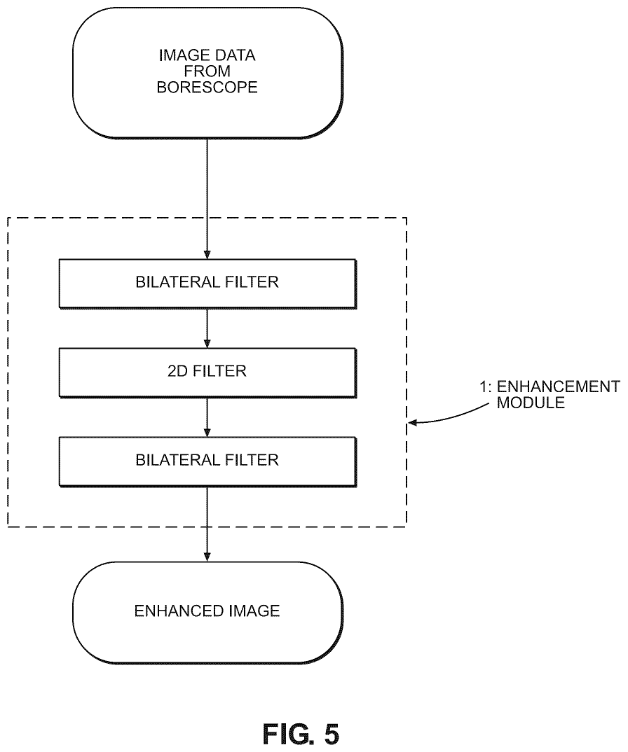

Referring to FIGS. 1-8, a technician pushes the digital borescope 200 through a working channel 114 of the medical scope 100. The (video RGB) image data produced from the pixels of the image sensor 214 of digital borescope 200 is sent along a network by which it is accessed in real time by deep learning tool 310. The deep learning tool 310 may also enhance the image data (1: Enhancement Module in FIGS. 4 and 5), i.e., in effect produce enhanced images of the standard definition images produced by the digital borescope 200. (S800)

The Detection module of the deep learning tool 310 has a convolutional neural network pre-trained on a data set of images of surface anomalies along channels of medical scopes like that through which the digital borescope 200 is pushed. And the convolutional neural network is configured for semantic segmentation, as exemplified by the flowchart and architecture shown in and described with reference to FIGS. 6A, 6B and 7. The image data is processed including by semantic segmentation only along a context path. Other processes include polygon extraction, tracker predictions and image fusion as respectively executed using the Suzuki contour algorithm, the Tracking module and the fusion algorithm shown in and described with reference to FIG. 4. (S810)

Indicia of surface anomalies along the length of the working channel 114 is output based on results of the processing (S820). For example, the indicia comprise data of surface anomalies, output by the Tracking module (FIG. 4) after having been subjected to the fusion algorithm, and can be used to generate graphics using the graphics output module 310c (FIG. 3). The data used for this purpose can also be stored in a memory and may be used to generate reports bearing on the inspection process, described in further detail below. Respective stored data may also be used to update the learning algorithm of the training module 310d (FIG. 3). Due to essentially no spatial information of the pixels in the image data being encoded in the segmentation mask produced by the Detection module, the entire process can be executed at a rate of no more than about 30 frames per second to detect and recognize any of various predetermined types of surface anomalies along the surface of the working channel 114 with a high degree of reliability.

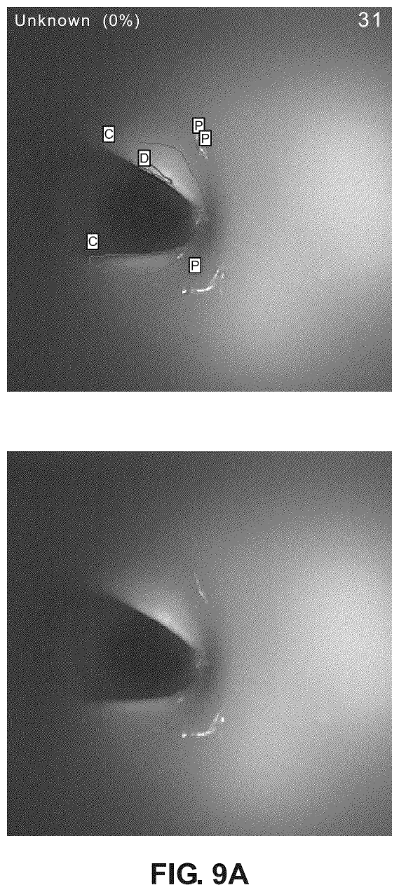

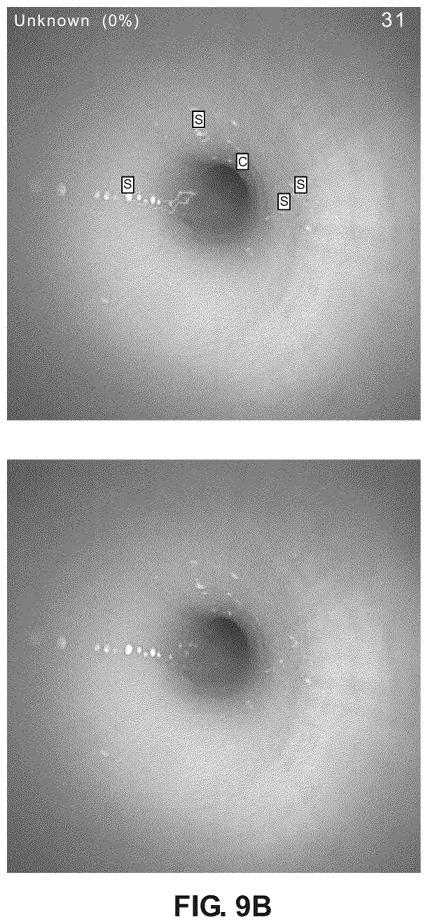

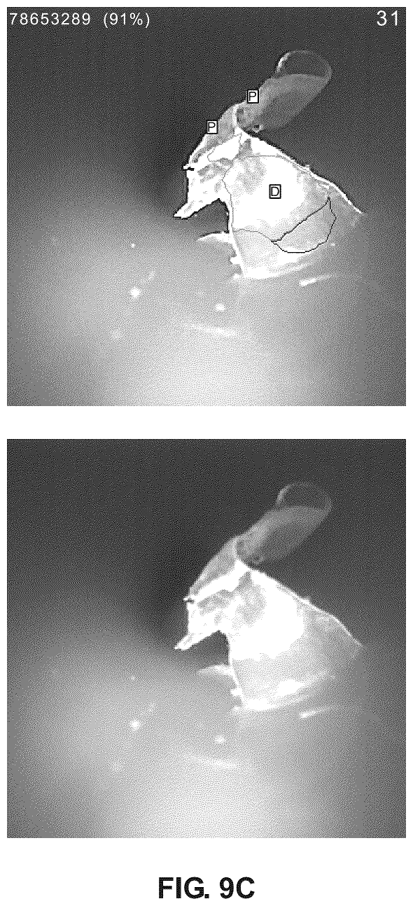

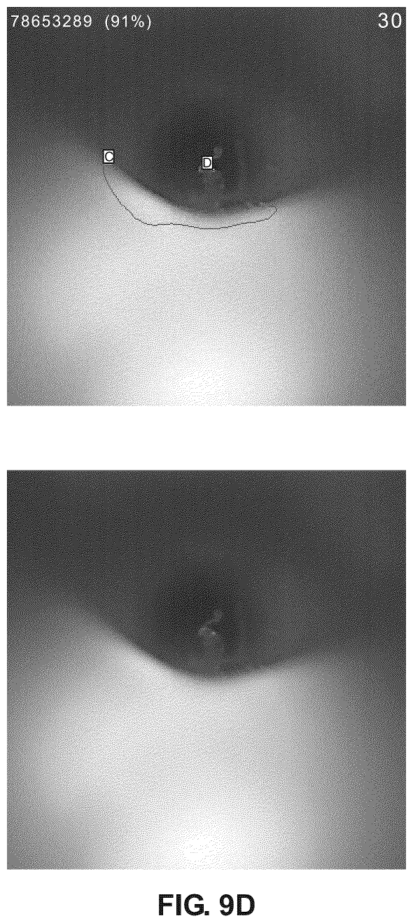

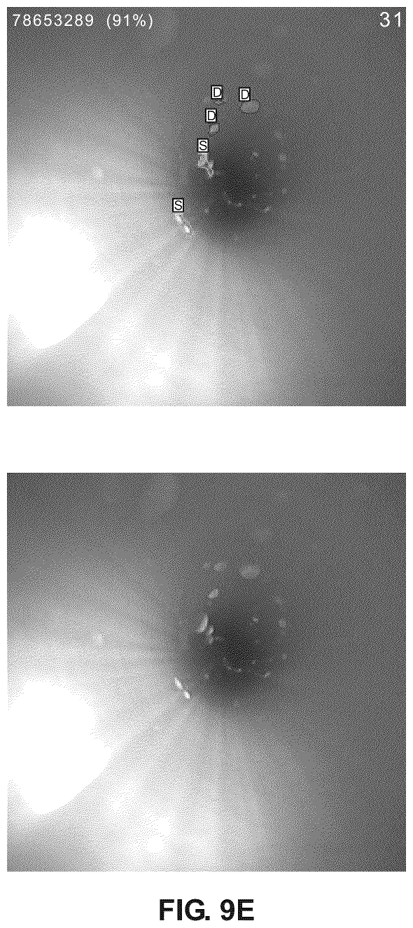

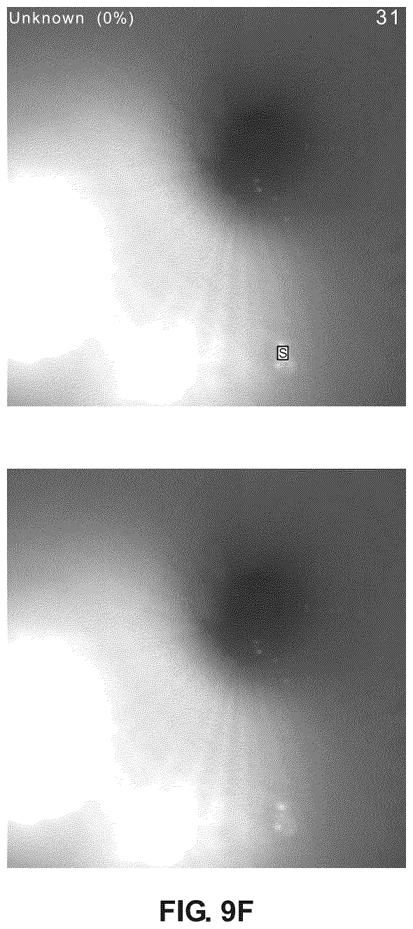

Thus, in what is essentially a time-neutral manner with respect to the outputting of images by the digital borescope 200, indicia of surface anomalies along the working channel 114 can be displayed on visual display unit 320 and reports can be automatically generated. The indicia may be in the form of enhanced images of the working channel 114 augmented with graphics overlaid on or near the surface anomalies (see FIGS. 9A and 9B and the description thereof below). The indicia may also comprise or consist of a table showing the instances and types of surface anomalies detected. Thus, the indicia may be analyzed by a technician to determine a course of action to be taken with respect to the medical scope, e.g., acceptable for use, to be re-processed or repaired, to be discarded as damaged beyond repair, etc.

The time neutrality referred to above can also apply to the performing of the following tasks. The output of the AI image recognition system 300 can be used to create a work flow by which usage of medical scopes tested is optimized, reducing turnover time associated with reprocessing. Indicators of foreign matter derived from the output, such as droplets, degree, organic matter, or brushes, can be used to score the cleaning process, such as the enzymatic cleaning process. Indicators of mechanical damage derived from the output, such as scratches, peeling, crimping, can be used to generate reports or emails associated with the repair process, such as emailing a department head for approval to take a medical scope out of service and send it off for repairs, or even emails to the repair service operator informing them of the nature and extent of necessary repairs. The same can be used to determine false positives or negatives and obviously, as referred to above, update the learning algorithm 310d. Because the detection and identification of surface anomalies occurs in real time with the attendant advantages of sensitivity and reliability regardless of the speed at which a borescope is pushed through the channel, these reports, emails, etc. can be generated in a time neutral manner.

FIGS. 9A-9F show samples of results of carrying out the method of FIG. 8 using the AI image recognitions system 300 (FIGS. 3-7) according to the present technology.

In the figures, the results that may be displayed on display 320 are the upper images in each figure, and the lower images show the corresponding images captured by the borescope 200 and accessed by the system as input. In this example, the indicia of the surface anomalies are provided as graphics overlayed on the processed images at the locations of the anomalies.

The graphics in this example include letter marks identifying the surface anomaly by class: “s” for scratch, “p” for peeling, “d” for droplet, “c” for crimp and so on, as well as enhanced outlines of the edge boundaries of the surface anomalies. Therefore, these lettermarks could readily be tabulated and displayed on the GUI of the visual display unit 320 in table form as further indicia of the surface anomalies.

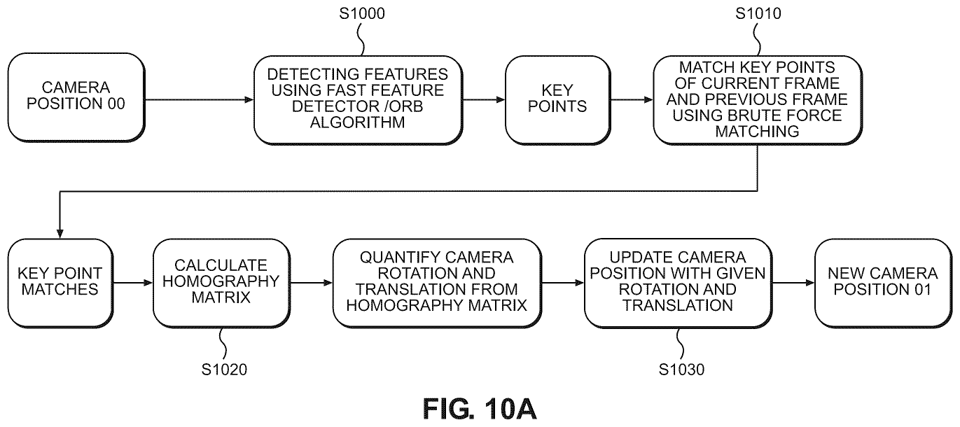

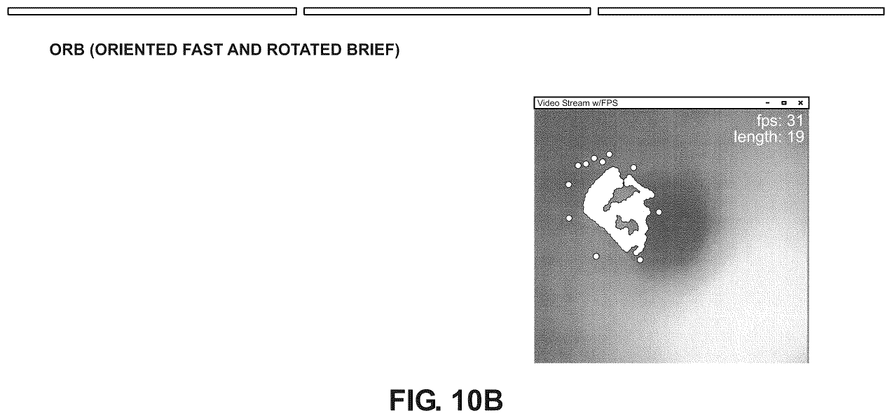

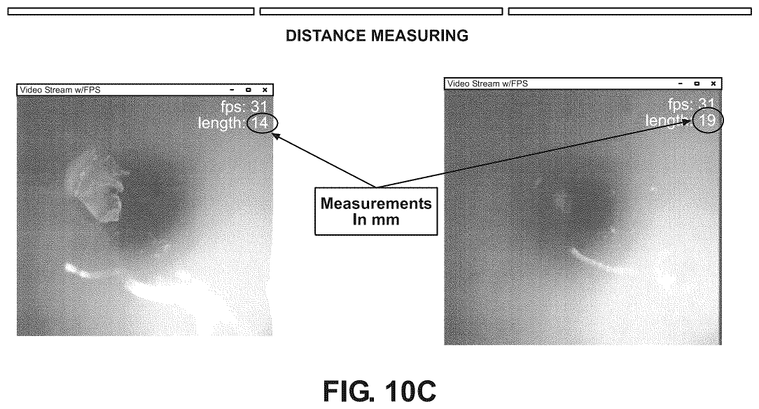

FIGS. 10A-C show an example of a visual odometry process for use in the present technology. Basically, this process allows the relative position of the video camera (image sensor 214 in FIG. 2, for example) in the working channel of the medical scope (e.g., instrument channel 114) to be determined from keypoints of images captured as the video camera is translated (pushed) through the working channel and in some instances, rotated as well. Rotation of the video camera is well-suited to capturing images of surface anomalies which may occur along only a select circumferential portion of the working channel at a given location along the length of the channel.

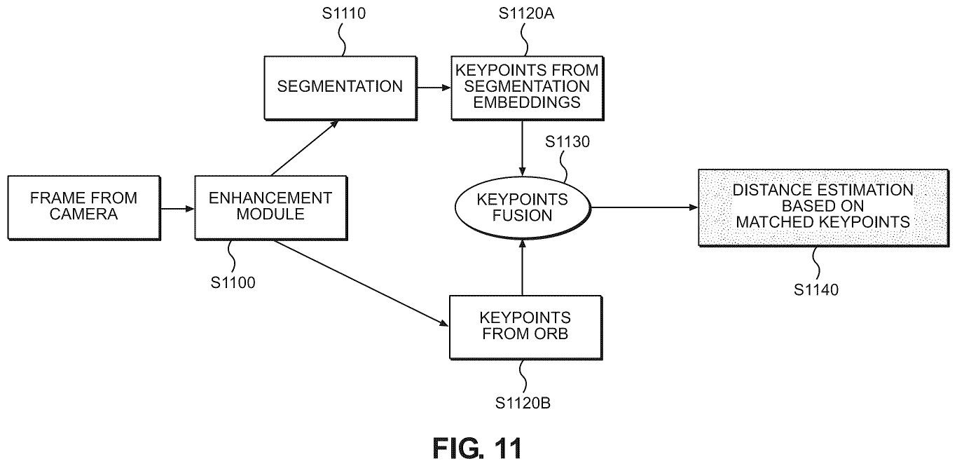

By combining a visual odometry tool/process with the inspection system/method of the present technology, coordinates of features along the length and circumference of the surface of the working channel can be determined, i.e., the features can be “mapped” relative to the working channel so as to be advantageously recallable. And by extension, as will be described later on with respect to FIG. 11, coordinates of anomalies along the surface of the working channel can be determined and used to estimate their distance from the end of the working channel so that again, the locations of the anomalies can be recalled.

Referring back to FIG. 10A, the initial block shows the video camera at an arbitrary position “00” as it is being pushed through the working channel. At position “00” the video camera is capturing a particular frame within the working channel. At S1000, keypoints in the image being captured by the camera (the current frame) are detected by processing the frame using an ORB (Oriented FAST and Rotated BRIEF) algorithm, known per se. To this end, the AI image recognition system 300 (FIG. 3) and, in particular, the deep learning tool 310 thereof, may include a memory (non-transitory CRM such as a RAM) storing the ORB algorithm, and a processing unit of the system connected to the memory serves to execute the ORB algorithm. The keypoints are specific points of interest in the image, which serve as a descriptor of the image. The keypoints are stored in a memory of the AI image recognition system 300, which may be the same memory that stores the training images or a discrete memory dedicated for storing the keypoints, and operatively connected to the processor. FIG. 10B shows an example of an image of a working channel in which the keypoints in the image or highlighted.

More specifically, FAST refers to Features from Accelerated and Segments Test, and selects pixel(s) of the image as keypoint(s) based on whether a certain number of surrounding pixels are darker or brighter than it. Since the FAST technique for selecting keypoints from an image is well known it will not be described further in detail. BRIEF stands for Binary Robust Independent Elementary Feature. BRIEF takes all of the keypoints extracted using the FAST algorithm and converts them into binary feature vectors so that together they can represent an object. Binary features vectors are feature vectors that only contain a 1 and a 0. In BRIEF, each keypoint is represented by a feature vector which is a 128-512 bits string.

The ORB algorithm can be programmed in Python as described in OpenCV under the heading “ORB (Oriented FAST and Rotated BRIEF)”.

Example of Simple Code for ORB in Python
import numpy as np
import cv2 as cv
from matplotlib import pyplot as plt
img=cv.imread(‘simple.jpg’, cv.IMREAD_GRAYSCALE)
#Initiate ORB detector
orb=cv.ORB_create( )
#find the keypoints with ORB
kp=orb.detect(img,None)
#compute the descriptors with ORB
kp, des=orb.compute(img, kp)
#draw only keypoints location,not size and orientation
img2=cv.drawKeypoints(img, kp, None, color=(0,255,0), flags=0)
plt.imshow(img2), plt.show( )

The keypoints detected using the ORB algorithm in the current frame are then matched (S2000) with keypoints detected from the previous frame also using the ORB algorithm. In this example, brute force matching is used. The algorithm can be programmed using Python provided in the popular OpenCV library under the topic heading, “Feature Matching”.

Example of Programming Brute-Force Matching with ORB descriptors in Python

1^{st} Step: load images and find descriptors
import numpy as np
import cv2 as cv
import matplotlib.pyplot as plt
img1=cv.imread(‘box.png’,cv.IMREAD_GRAYSCALE) #queryImage
img2=cv.imread(‘box_in_scene.png’,cv.IMREAD_GRAYSCALE) #trainImage
#Initiate ORB detector
orb=cv.ORB_createo
#find the keypoints and descriptors with ORB
kp1, des1=orb.detectAndCompute(img1,None)
kp2, des2=orb.detectAndCompute(img2,None)

2^{nd} Step: create a BFMatcher object with distance measurement
#create BFMatcher object
bf=cv.BFMatcher(cv.NORM_HAMMING, crossCheck=True)
#Match descriptors.
matches=bf.match(des1,des2)
#Sort them in the order of their distance.
matches=sorted(matches, key=lambda x:x.distance)
#Draw first 10 matches.
img3=cv.drawMatches(img1,kp1,img2,kp2,matches[:10],None,flags=cv.DrawMatchesFlags_NOT_DRAW_SINGLE_POINTS)
plt.imshow(img3),plt.show( )

Next, a transformation between the current and previous frames is calculated (S1020). Basically, this step compares the coordinates of the keypoints between the current and previous frames. By matching keypoints between the frames, the distance that the video camera has moved (translated along the working channel or both translated along and rotated relative to the working channel) can be quantified. And example of the current and previous frames and calculation of the translation of the video camera therebetween is shown in FIG. 10C.

In the illustrated example, a homography transform is used to create a “homography matrix” of the matching keypoints. The homography matrix in this application represents the relationship between the two frames. The homography matrix allows the distance between the frames to be determined and this distance can be used as the amount of translation (and rotation) of the camera along the working channel from the time the previous frame was captured to the time the current frame is being captured. As with the ORB algorithm, the transform is stored in a memory (a non-transitory CRM) of the AI image recognition system 300 and is executed under the control of the processing unit of the system. Alternatively, simple Euclidean geometry can be applied to the matching keypoints to calculate the mean distances between the matching keypoints, respectively. And by virtue of triangulation these mean distance can be used to determine the amount of translation (and rotation) of the camera along the working channel.

Next, the relative position of the video camera in the working channel is updated (S1030) by the amount of translation (and rotation) of the video camera determined in S1020 to obtain the “New Camera position 01”. The process is then repeated for each resulting “New Camera position 01.” In this way, the images having certain keypoints can be correlated with the both the relative position of the video camera within the working channel.

FIG. 11 illustrates how the system of FIGS. 3 and 4 and method of FIG. 10A may be adopted for use in estimating the distance of surface anomalies from the end (distal or proximal) of the working channel of the medical scope. In S1100, the frame (image) from the video camera is enhanced to produce enhanced image data. The enhancement may be carried out by the Image enhancement module shown and described with reference to FIG. 4. The enhanced image data is processed in parallel. In one part, the enhanced image data is subjected to semantic segmentation (S1110) by the Detection module (shown and described with reference to FIG. 4). As part of this process embeddings (vectors) are created by the machine learning tool, providing meaningful data by which surface anomalies are detected by the deep learning tool 310. In S1120A, keypoints of the segmentation embeddings are detected. At the same time, keypoints in the enhanced image are detected using the ORB shown in and described with reference to S1000 in FIG. 10A.

Next, the keypoints from the segmentation embeddings and the keypoints from the ORB algorithm are fused (S1130). Using a process flow similar to that in FIG. 10A, the fused keypoints are used to update the camera position and thus provide an estimation of the distance of the surface anomaly from the end of the medical scope having the opening through which the video camera was inserted.

In some examples, each time a surface anomaly is detected, a tag is assigned to it. For example, if ten scratches are detected in the surface of a working channel of a medical scope, they are tagged and stored in the memory of the deep learning tool 310 (FIG. 3) and retrieved for future reference. The memory may be the same memory 330 described as including the training images or may be a discrete memory dedicated for this purpose. Having this information on hand, i.e., identifying or “tagging” the surface anomalies according to their relative locations (longitudinal and circumferential) in the working channel, provides the following benefits.

During any inspection process, the video camera can be translated back and forth or rotated within the working channel without “counting” the same surface anomaly a second time. Thus, multiple passes over the same length of the working channel can be used to confirm and hence, improve the accuracy of results instead of yielding false data of numbers of discrete surface anomalies. Or, the technician conducting the inspection process may want to revisit a particular surface anomaly during the inspection process for any number of reasons, such as for comparing and contrasting different surface anomalies along the length of the working channel.

Likewise, it might be desirable to re-inspect a medical scope after subsequent use, repair or reprocessing. In these cases, it would be invaluable to be able to return the video camera precisely to the location of a previously detected surface anomaly without seeing it as a newly created surface anomaly. For example, in the case of tagging a scratch along the surface of the working channel, returning the video camera to the precise location of the tagged scratch could be used to track the progression of the scratch, i.e., whether the scratch has become deeper or longer. Or, one may return to and keep looking at the same stain on the surface of the working channel to ascertain whether the stain is getting bigger, worse, and/or has bacteria growing on it. By performing this process multiple times, predictable algorithms may be developed to determine the remaining useful life of the medical scope. In the case of repair or reprocessing, the efficacy of the reprocessing or repair process could be evaluated by seeing its effect on the tagged surface anomalies.

According to still another example of the present technology, the deep learning tool 310 (FIG. 3) is trained to recognize whether the video camera is being inserted into the proximal or distal end of the working channel of a medical scope (refer back to FIGS. 1A and 1B showing an example of a medical scope has openings delimiting the distal and proximal ends of the working channel 114). In one example, the deep learning tool 310 of the AI image recognition system 300 is trained on images of the distal and proximal ends of the working channels of medical scopes so that the system automatically recognizes which end of the working channel that the video camera of the digital borescope 200 is being inserted into. In another example, the working channels of medical scopes can be provided with “marks” at certain preset distances from the open ends of the working channels. These “calibration” marks are also configured to serve as identifiers as to whether the ends closest thereto are the distal or proximal ends. In this example, the deep learning tool 310 of the AI image recognition system 300 is trained on images of the calibration marks. And because the digital borescope 200 contains a light source, the images of the calibration marks can be captured and based on the training, the AI image recognition system 300 can automatically recognize which end of the working channel that the video camera of the digital borescope 200 is being inserted into.

In these examples, all of the relative positions of the surface anomalies can be recorded as distances from that end of the working channel—distal or proximal—recognized as having initially received the video camera. Therefore, the personnel conducting the inspections or re-inspections are free to insert the video camera through either the distal or proximal end of the working channel, i.e., without any need to record that information as the reference end for the estimated positions of the detected surface anomalies. In any case, a report may be automatically generated in the form of a document showing where the detected surface anomalies were located relative to the (automatically) detected end of the working channel and the time the anomalies were observed relative to a start time of the inspection process (e.g., the time the video camera was inserted into the opening of the working channel or the time the video camera arrived at the calibration mark within the channel).

Still further, as was clear from the description of FIG. 3, a system according to the present technology may be implemented through cloud computing, in which a complex, high-powered computing device (GPU) including the deep learning tool 310 communicates through the cloud with portable computing devices, such as tablets. In this way, reporting and viewing of surface anomalies of medical scopes can be live-streamed or later uploaded to relatively inexpensive portable computing devices over a network. This avoids the need for organizations to make investments in the relatively expensive equipment necessary detect surface anomalies and conduct the visual odometry as described above.

As is clear from the description above, the present technology offers an enhancement of the speed at which AI object detection can take place, at least in the context of detecting surface anomalies in medical scopes, and implements visual odometry to provide positional data of the camera from which the relative locations of the surface anomalies may be determined. Thus, the present technology can reduce labor costs associated with the inspection of channels of medical scopes, reduce downtime, facilitate protocols for repair, evaluate the efficacy of repair, predict the remaining useful life of medical scopes, etc. Furthermore, although the present technology has been described above in detail with respect to various embodiments and examples thereof, the technology may be embodied in many different forms to implement the present invention. Thus, the present invention should not be construed as being limited to the embodiments and their examples described above. Rather, these embodiments and examples were described so that this disclosure is thorough, complete, and fully conveys the present invention to those skilled in the art. Thus, the true spirit and scope of the present invention is not limited by the description above but by the following claims.

What is claimed is:

1. A method for use in evaluating a state of a medical scope having a working channel, the method comprising:
accessing, at an artificial intelligence (AI) image recognition system including a deep learning tool, image data of video of the working channel of a medical scope produced by a video camera as the video camera is pushed along a length of the working channel beginning at an end of the working channel,
the deep learning tool having a convolutional neural network, the convolutional neural network configured for semantic segmentation along a context path, and the convolutional neural network being trained on a data set of images of at least one type of surface anomaly along channels of medical scopes similar to that through which the borescope is pushed;
processing the image data using the AI image recognition system to both detect surface anomalies of said at least one type at locations along the length of the working channel and calculate for each of the surface anomalies detected an estimation of a distance between the detected surface anomaly and said end of the working channel,
the processing including semantic segmentation of the image data by the convolutional neural network along only the context path in the AI image recognition system once the data of video images is accessed by the AI image recognition system to detect the presence of surface anomalies along the working channel, extracting keypoints of images taken by the video camera within the working channel, and using the keypoints to determine amounts of translation of the video camera within the working channel; and
producing data of the types of surface anomalies detected and their relative locations within the working channel based on results of the processing.

2. The method as claimed in claim 1, wherein the processing of the image data comprises downsampling image data in the deep learning tool in a series of downsampling processes, applying global average pooling at a tail end of the downsampling processes, and subsequently upsampling only output derived from the global pooling.

3. The method as claimed in claim 1, wherein the semantic segmentation assigns semantic labels to pixels outputting image data of edges of features along the channel, and the processing further includes:
a polygon extraction process that integrates the edges with polygons to produce data of the type of the surface anomaly,
a tracking process that tracks results of the semantic segmentation and polygon extraction processes between frames to discriminate surface anomalies from one another, and
a tracker and detection fusion process that outputs data of numbers and type of surface anomalies.

4. The method as claimed in claim 1, wherein the extracting of keypoints comprises applying an oriented FAST (features from accelerated and segments test) and rotated BRIEF (binary robust independent elementary feature) algorithm to the image data.

5. The method as claimed in claim 4, wherein the using of the keypoints comprises matching the keypoints of a current frame captured by the video camera with the keypoints of a previous frame captured by the video camera, and using the keypoint matches to determine an amount of translation of the camera between the frames.

6. The method as claimed in claim 5, wherein the matching of the keypoints comprises brute force matching.

7. The method as claimed in claim 1, wherein the processing further comprises enhancing the image data before the semantic segmentation of the image data.

8. The method as claimed in claim 7, wherein the extracting of keypoints comprises applying an oriented FAST (features from accelerated and segments test) and rotated BRIEF (binary robust independent elementary feature) algorithm (ORB algorithm) to the image data after it has been enhanced.

9. The method as claimed in claim 8, wherein the processing comprises fusing keypoints from embeddings of the semantic segmentation of the enhanced image data with the keypoints from the applying of the ORB algorithm to the enhanced image data to estimate distances of the detected surface anomalies from the end of the working channel.

10. The method as claimed in claim 1, further comprising an AI image recognition process of recognizing whether the end of the working channel through which the video camera is inserted is a distal or proximal end of the working channel,
the amounts of translation of the video camera within the working channel are determined from said distal or proximal end of the working channel; and
the producing of data includes estimating a distance of each of the anomalies detected from said distal or proximal end of the working channel.

11. A method for use in evaluating a state of a medical scope having a working channel, the method comprising:
pushing a video camera, including an image sensor having an array of pixels, through an end of the working channel and along a length of the working channel to capture images of a length of the working channel and output image data representative of the images;
transmitting image data from each of the pixels of the image sensor of the video camera to an artificial intelligence (AI) image recognition system including a deep learning tool having a convolutional neural network, the convolutional neural network being trained on a data set of images of surface anomalies along channels of medical scopes similar to that through which the video camera is being pushed, and the deep learning tool being configured for semantic segmentation along a context path;
processing image data produced from the pixels of the image sensor of the video camera using the AI image recognition system to both detect surface anomalies along the length of the working channel and calculate for each of the surface anomalies detected an estimation of a distance between the detected surface anomaly and said end of the working channel,
the processing including semantic segmentation of the image data by the convolutional neural network along only a context path in the deep learning tool of the AI image recognition system once the image data is received by the AI image recognition system to detect the presence of surface anomalies along the working channel, extracting keypoints of images taken by the video camera within the working channel, and using the keypoints to determine amounts of translation of the video camera within the working channel; and
producing data of the types of surface anomalies detected and their relative locations within the working channel based on results of the processing.

12. The method as claimed in claim 11, wherein the processing of the image data comprises downsampling image data in the deep learning tool in a series of downsampling processes, applying global average pooling at a tail end of the downsampling processes, and subsequently upsampling only output derived from the global pooling.

13. The method as claimed in claim 11, wherein the semantic segmentation assigns semantic labels to pixels outputting image data of edges of features along the channel, and the processing further includes:
a polygon extraction process that integrates the edges with polygons to produce data of the type of the surface anomaly,
a tracking process that tracks results of the semantic segmentation and polygon extraction processes between frames to discriminate surface anomalies from one another, and
a tracker and detection fusion process that outputs data of numbers and type of surface anomalies.

14. The method as claimed in claim 11, wherein the extracting of keypoints comprises applying an oriented FAST (features from accelerated and segments test) and rotated BRIEF (binary robust independent elementary feature) algorithm to the image data.

15. The method as claimed in claim 14, wherein the using of the keypoints comprises matching the keypoints of a current frame captured by the video camera with the keypoints of a previous frame captured by the video camera, and using the keypoint matches to determine an amount of translation of the camera between the frames.

16. The method as claimed in claim 15, wherein the matching of the keypoints comprises brute force matching.

17. The method as claimed in claim 11, wherein the processing further comprises enhancing the image data before the semantic segmentation of the image data.

18. The method as claimed in claim 17, wherein the extracting of keypoints comprises applying an oriented FAST (features from accelerated and segments test) and rotated BRIEF (binary robust independent elementary feature) algorithm (ORB algorithm) to the image data after it has been enhanced.

19. The method as claimed in claim 18, wherein the processing comprises fusing keypoints from embeddings of the semantic segmentation of the enhanced image data with the keypoints from the applying of the ORB algorithm to the enhanced image data to estimate distances of the detected surface anomalies from the end of the working channel.

20. The method as claimed in claim 11, further comprising an AI image recognition process of recognizing whether the end of the working channel through which the video camera is inserted is a distal or proximal end of the working channel,
the amounts of translation of the video camera within the working channel are determined from said distal or proximal end of the working channel; and
the producing of data includes estimating a distance of each of the anomalies detected from said distal or proximal end of the working channel.
